(19) United States
(12) Patent Application Publication
(10) Pub. No.: US 2024/0164410 A1
(43) Pub. Date: May 23, 2024

(54) A STABLE, EMULSION-FORMING LIQUID COMPOSITION COMPRISING AMINO ACIDS

(71) Applicant: BrainLuxury Inc., Brooklyn, NY (US)

(72) Inventors: Axel Bouchon, Kleinmachnow (DE); Herman Schützinger, Salzburg (AT)

(21) Appl. No.: 18/552,070

(22) PCT Filed: Mar. 25, 2022
(86) PCT No.: PCT/US2022/022027

§ 371 Date: Sep. 22, 2023

Related U.S. Application Data
(60) Provisional application No. 63/165,887, filed on Mar. 25, 2021.

(51) Int. Cl. A23L 2/06; A23L 2/52; A61K 9/00; A61K 9/107; A61K 31/05; A61K 31/198; A61K 31/201; A61K 31/355; A61K 31/375; A61K 31/381; A61K 31/405; A61K 31/593; A61K 33/06; A61K 36/02; A61K 36/55; A61K 36/889; A61P 25/26

(52) U.S. Cl. CPC A23L 2/06; A23L 2/52; A61K 9/0095; A61K 9/107; A61K 31/05; A61K 31/198; A61K 31/201; A61K 31/355; A61K 31/375; A61K 31/381; A61K 31/405; A61K 31/593; A61K 33/06; A61K 36/02; A61K 36/55; A61K 36/889; A61P 25/26

(57) ABSTRACT

The disclosure is in part directed to shelf-stable, emulsion-forming liquid compositions, liquid beverage compositions, and fatty acid vesicle-forming liquid compositions for increased neurosystem uptake of amino acids present in the compositions when consumed by a subject.

### BACKGROUND

Proteins commonly found in foods do not necessarily provide an amino acid composition that provides the amino acid requirements or needs of a mammal, such as a human, in an efficient manner. For example, it can be difficult to attain a meaningful threshold of certain amino acids in the brain. The brain is known to be acutely sensitive to changes in plasma amino acid concentrations within the physiologic range. An example of this sensitivity is the inhibition in cerebral protein synthesis caused by hyperphenylalaninemia and the reversal of this inhibition by the co-administration of other large neutral amino acids that compete with phenylalanine for transport into the brain from blood.

Further, certain amino acids play key roles in the nervous system (e.g., the central nervous system), for example, by serving as biosynthetic precursors for neurotransmitters or by serving as neurotransmitters themselves. For example, tryptophan may affect serotonin function, while tyrosine and/or phenylalanine may affect dopamine function. Typically, however, these amino acids often do not have quick availability to the brain and other neurosystems and therefore can be difficult to use, e.g., to provide assistance for healthier sleep and mood. For example, tryptophan, tyrosine, and phenylalanine each require a carrier protein for transport across the blood-brain barrier. If the amount of tryptophan entering the brain is limited, the amount of serotonin and melatonin being synthesized in the brain is also limited. Such reduced levels of serotonin in the brain are often manifested, for example, by alterations in sleep, mood, and overall well-being, as well as being associated with depression.

Mixtures comprising essential amino acids (e.g., tryptophan) have been used that hydrolyze a protein with relatively high levels of essential amino acids, such as whey protein, and/or by combining free amino acids in a mixture that optionally also includes a hydrolyzed protein such as whey. However, mixtures of this type may have a bitter taste, undesirable mouthfeel, the potential to create gastrointestinal upset and are poorly soluble, and may be deemed unsuitable or undesirable for certain uses.

Accordingly, it would be useful in certain situations to have compositions for supplying proteins that may e.g., facilitate amino acid delivery upon mammalian consumption, and may e.g. support natural sleep and that are also substantially shelf stable and palatable.

### SUMMARY

The present disclosure is directed, in one embodiment, to an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, phenylalanine, and tyrosine; and b) a fatty acid composition comprising at least one omega-9 fatty acid, at least one omega-6 fatty acid, and at least three omega-3 fatty acids; wherein the aqueous liquid composition and the fatty acid composition form an amino acid/fatty acid buffer system allowing the creation of a fatty acid vesicle-based emulsion when the aqueous liquid composition and the fatty acid composition are shaken together.

For example, disclosed herein is an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, phenylalanine, and tyrosine; and b) a fatty acid composition comprising at least three omega-3 fatty acids: wherein the aqueous liquid composition and the fatty acid composition form an amino acid/fatty acid buffer system allowing the creation of a fatty acid vesicle-based emulsion when the aqueous liquid composition and the fatty acid composition are shaken together.

Also disclosed herein is a liquid beverage composition comprising one or more omega-6 fatty acids; one or more omega-9 fatty acids; three or more omega-3 fatty acids, wherein the weight ratio of omega-6 fatty acids:omega-9 fatty acids:omega-3 fatty acids is about 1:about 1.7 to about 2.3:about 5.7 to about 6.3:L-tryptophan; L-phenylalanine; and an effective amount of a food-compatible acidic agent; wherein the liquid beverage composition has a pH of about 3.2 to about 3.6 as measured at 20° C.

Further disclosed herein is a liquid beverage composition comprising flaxseed oil, algae oil, a plant-derived oil, L-tryptophan, and L-phenylalanine; wherein the omega-6 fatty acids, omega-9 fatty acids and omega-3 fatty acids present in the flaxseed oil, algae oil, and plant derived oil are in a weight ratio of omega 6 fatty acids:omega 9 fatty acids:omega 3 fatty acids of about 1:about 1.7 to about 2.3:about 5.7 to about 6.3.

Also disclosed herein is a liquid beverage composition comprising flaxseed oil, algae oil, a plant-derived oil. L-tryptophan, and L-phenylalanine; wherein the eicosapentaenoic acid, docosahexaenoic acid, and α-linoleic acid present in the flaxseed oil, algae oil, and plant-derived oil combined are present in a weight ratio of eicosapentaenoic acid:docosahexaenoic acid:α-linoleic acid of about 1:about 1.8 to about 2.2:about 3.5 to about 4.1.

Additionally disclosed herein is a fatty acid vesicle-forming liquid composition comprising, per 60 ml of the liquid composition:about 1-5 ml flaxseed oil; about 1-5 ml algae oil; about 1-5 ml of a plant-derived oil, wherein the plant-derived oil is selected from the group consisting of olive oil, avocado oil, coconut oil, and mixtures thereof; about 150 to about 650 mg L-phenylalanine; about 150 to about 450 mg L-tryptophan; and about 47-59 ml of a food-compatible aqueous solution with a pH of about 3.0 to about 3.9 at 20° C.; wherein when the liquid composition is shaken for 10 seconds or longer, the creation of fatty acid vesicles occurs.

Additionally disclosed herein is a fatty acid vesicle-forming liquid composition comprising, per 60 ml of the liquid composition:about 2 ml flaxseed oil; about 2 ml algae oil; about 2 ml of a plant-derived oil, wherein the plant-derived oil is selected from the group consisting of olive oil, avocado oil, coconut oil, and mixtures thereof:about 400 mg L-phenylalanine; about 200 mg L-tryptophan; and about 54 ml of a food-compatible aqueous solution with a pH of about 3.0 to about 3.9 at 20° C.; wherein when the liquid composition is shaken for 10 seconds or longer, the creation of fatty acid vesicles occurs.

Also provided herein, for example, is method of enhancing brain performance in a human subject in need thereof, comprising administering to the human subject an effective amount of a composition disclosed herein, for example, a disclosed emulsion-forming liquid composition, a disclosed liquid beverage composition, or a disclosed fatty acid vesicle-forming liquid composition.

Further disclosed herein, for example, is a method of increasing energy levels, productivity, and/or creativity in a human subject in need thereof, comprising administering to the human subject an effective amount of a composition disclosed herein, for example, a disclosed, emulsion-forming liquid composition, a disclosed liquid beverage composition, or a disclosed fatty acid vesicle-forming liquid composition.

### BRIEF DESCRIPTION OF THE DRAWINGS

FIG. 1A depicts a group analysis on sleeping pill reduction in a study population before and after consumption of a disclosed liquid composition.

FIG. 1B depicts a group analysis of self-assessed energy levels during the day in a study population before and after consumption of a disclosed liquid composition.

FIG. 2A depicts a group analysis of self-assessed productivity levels in a study population before and after consumption of a disclosed liquid composition.

FIG. 2B depicts a group analysis of self-assessed creativity levels in a study population before and after consumption of a disclosed liquid composition.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. All ranges recited herein include the endpoints, including those that recite a range “between” two values.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

The term “effective amount” means the amount of a disclosed liquid composition required to achieve a desired biological effect in a human cell, tissue, organ, or system. The term “subject” refers to a human subject.

As used herein, a “shelf-stable” liquid composition refers to a disclosed liquid composition that has been processed or packaged in order to withstand prolonged storage.

As used herein, the terms “amphiphilic amino acids” and “amphipathic amino acids” are used interchangeably and refer to those amino acids having both polar and nonpolar characteristics, e.g., those amino acids having both hydrophilic and hydrophobic properties.

As used herein, the term “plant-derived oil” refers to an oil derived from a plant source as opposed to an animal source or a petroleum source.

### Compositions

Disclosed herein in an embodiment is an emulsion-forming liquid composition, that may, for example, increase neurosystem uptake of certain amino acids present in the disclosed emulsion-forming liquid composition when consumed in emulsion form by a subject, comprising: a) aqueous liquid composition having a pH of about 3.0 to about 3.9 at 20° C., where the aqueous liquid composition comprises at least two amino acids each independently selected from the group consisting of tryptophan, 5-hydroxytryptophan, phenylalanine, and tyrosine; and b) a fatty acid composition comprising at least one omega-9 fatty acid, at least one omega-6 fatty acid; and at least three omega-3 fatty acids. The aqueous liquid composition and the fatty acid composition, when shaken together, form an amino acid/fatty acid buffer system allowing the creation of a fatty acid vesicle-based emulsion. Contemplated herein is a composition that where the aqueous liquid composition and the fatty acid composition are contained within one container. In other embodiments, the aqueous liquid composition and the fatty acid composition may be contained within separate containers and then combined before the emulsion-forming liquid composition is shaken.

The aqueous liquid composition portion of the disclosed liquid composition may be a fruit juice. Contemplated fruit juice may be, but not limited to, apple juice, apricot juice, blackberry juice, black current juice, blueberry juice, boysenberry juice, calamansi juice, cantaloupe juice, cherry juice, clementine juice, cranberry juice, gooseberry juice, grape juice, grapefruit juice, guava juice, honeydew juice, lime juice, lime juice such as kaffir lime juice and/or key lime juice, kiwifruit juice, kumquat juice, lemon juice, lingonberry juice, lychee juice, mandarin orange juice, mango juice, marionberny juice, mulberry juice, muskmelon juice, nectarine juice, orange juice, papaya juice, passion fruit juice, peach juice, pear juice, persimmon juice, pineapple juice, plum juice, pomegranate juice, prune juice, raspberry juice, red currant juice, strawberry juice, tangerine juice, watermelon juice, winter melon juice, and/or combinations thereof. In some embodiments, the fruit juice comprises orange juice. In other embodiments, the fruit juice comprises lemon juice. In further embodiments, the fruit juice comprises orange juice and lemon juice. Contemplated aqueous liquid compositions that form part of a disclosed composition may further include water, coconut water, flavored mineral water and/or other flavored water.

Contemplated aqueous liquid composition portions of the disclosed liquid composition may include two or more different amino acids each of which may independently be, but are not limited to, e.g., alanine, arginine, asparagine, aspartic acid, cysteine, glutamic acid, glutamine, glycine, histidine, isoleucine, lysine, methionine, phenylalanine, proline, selenocysteine, serine, threonine, tryptophan, 5-hydroxytryptophan, tyrosine, and valine. In other embodiments, the aqueous liquid composition comprises, for example, two or more essential amino acids each of which may independently be, but are not limited to, e.g., histidine, isoleucine, leucine, lysine, methionine, phenylalanine, threonine, tryptophan, and valine. In other embodiments, the aqueous liquid composition comprises, for example, two or more conditionally essential amino acids each of which may independently be, but are not limited to, e.g., arginine, cysteine, glycine, glutamine, proline, and tyrosine. In other embodiments, the aqueous liquid composition comprises, for example, two or more non-essential amino acids each of which may independently be, but are not limited to, e.g., alanine, aspartic acid, asparagine, glutamic acid, serine, and selenocysteine. In other embodiments, the aqueous liquid composition comprises, for example, two or more amphiphilic amino acids each of which may independently be, but are not limited to, e.g., glycine, lysine, phenylalanine, tryptophan, and tyrosine. In other embodiments, the aqueous liquid composition comprises two or more different amino acids each independently selected from the group consisting of tryptophan, 5-hydroxytryptophan, glycine, tyrosine, and phenylalanine. In other embodiments, the aqueous liquid composition comprises two or more different amino acids each independently selected from the group consisting of tryptophan, 5-hydroxytryptophan, tyrosine and phenylalanine. In other embodiments, the aqueous liquid composition comprises two or more different amino acids each independently selected from the group consisting of tryptophan, tyrosine, and phenylalanine. Amino acids present in the aqueous liquid composition portion of a disclosed liquid composition may each independently be an L-isomer or a mixture of D- and L-isomers.

In some embodiments, contemplated aqueous liquid composition portions of a disclosed liquid composition may include, for example, L-tryptophan and L-phenylalanine. In some embodiments, the L-tryptophan and L-phenylalanine are present in a weight ratio of about 1:about 1.7 to about 2.3. In other embodiments, the L-tryptophan and L-phenylalanine are present in a weight ratio of about 1:about 1.8 to about 2.2. In other embodiments, the L-tryptophan and L-phenylalanine are present in a weight ratio of about 1:about 1.9 to about 2.1. In other embodiments, the L-tryptophan and L-phenylalanine are present in a weight ratio of about 1:about 2.0. In some embodiments, a contemplated liquid beverage composition comprises, for example, about 150 to about 350 mg L-tryptophan, e.g., about 150 to about 300 mg L-tryptophan, e.g., about 150 to about 250 mg L-tryptophan, e.g., about 175 to about 225 mg L-tryptophan, e.g., about 200 mg L-tryptophan; and about 150 to about 450 mg L-phenylalanine, e.g., about 200 to about 450 mg L-phenylalanine, e.g., about 250 to about 450 mg L-phenylalanine, e.g., about 300 to about 450 mg L-phenylalanine, e.g., about 350 to about 450 mg L-phenylalanine, e.g., about 375 to about 425 mg L-phenylalanine, e.g., about 400 mg L-phenylalanine.

A contemplated emulsion-forming liquid composition may further include magnesium and/or a salt or hydrate thereof. For example, in some embodiments the emulsion-forming liquid composition further comprises magnesium L-ascorbate dihydrate.

A contemplated emulsion-forming liquid may further include α-lipoic acid, resveratrol, and/or creatine. For example, in some embodiments the emulsion-forming liquid composition further comprises α-lipoic acid. In other embodiments, the emulsion-forming liquid composition further comprises resveratrol. In other embodiments, the emulsion-forming liquid composition further comprises creatine. In other embodiments, the emulsion-forming liquid composition further comprises α-lipoic acid and resveratrol. In other embodiments, the emulsion-forming liquid composition further comprises α-lipoic acid and creatine. In other embodiments, the emulsion-forming liquid composition further comprises resveratrol and creatine. In other embodiments, the emulsion-forming liquid composition further comprises α-lipoic acid, resveratrol, and creatine. In some embodiments, the weight ratio of α-lipoic acid:resveratrol:creatine is, e.g., 1:about 1.7 to about 2.3:about 3.5 to about 4.0. In some embodiments, the weight ratio of α-lipoic acid:resveratrol:creatine is, e.g., 1:about 1.8 to about 2.2:about 3.6 to about 3.9. In some embodiments, the weight ratio of α-lipoic acid:resveratrol:creatine e.g., 1:about 1.9 to about 2.1:about 3.7 to about 3.8. In some embodiments, the weight ratio of α-lipoic acid:resveratrol:creatine is, e.g., 1:about 2.0:about 3.6 to about 3.8. In some embodiment, the weight ratio of α-lipoic acid:resveratrol:creatine e.g., 1:about 2.0 to about 2.1:about 3.7 to about 3.8. In some embodiments, the weight ratio of α-lipoic acid:resveratrol:creatine e.g., 1:about 2.0:about 3.7 to about 3.8. For example, the contemplated emulsion-forming liquid may further include about 100 to about 300 mg α-lipoic acid, e.g., about 125 to about 275 mg α-lipoic acid, e.g., about 150 to about 250 mg α-lipoic acid, e.g., about 175 to about 225 mg α-lipoic acid, e.g., about 200 mg α-lipoic acid; about 100 to about 300 mg resveratrol, e.g., about 125 to about 275 mg resveratrol, e.g., about 150 to about 250 mg resveratrol, e.g., about 175 to about 225 mg resveratrol, e.g., about 200 mg resveratrol; and about 500 to about 1000 mg creatine, e.g., about 550 to about 950 mg creatine. e.g., about 600 to about 900 mg creatine, e.g., about 650 to about 850 mg creatine, e.g., about 700 to about 800 mg creatine, e.g., about 750 mg creatine.

The fatty acid composition component may include a weight ratio of omega-6 fatty acids:omega 9-fatty acids:omega-3 fatty acids of about 1:about 1.7 to about 2.3:about 5.7 to about 6.3, and/or provide a weight ratio of eicosapentaenoic acid:docosahexaenoic acid:α-linoleic acid is about 1:about 1.8 to about 2.2:about 3.5 to about 4.1. Such fatty acid compositions may include one or more of, for example, flaxseed oil, algae oil, fish oil, and/or at least one plant-derived oil (e.g., coconut oil, avocado oil, and/or olive oil). Exemplary algae oils may be derived from algae found in marine or freshwater habitats. Exemplary algae oils may be derived from microalgae or macroalgae. Exemplary algae oils may include, for example, one or more of the following: brown algae oil, green algae oil, red algae oil, seaweed oil, kelp oil, rockweed oil, sargassum oil, Irish moss oil, coralline oil, dulse oil, sea lettuce oil, spirulina oil, chlorella oil, Schizochytrium sp, oil, and mixtures thereof. Exemplary plant-derived or plant-based oils may be derived from plants found in marine, freshwater or land habitats. Exemplary plant-derived or plant-based oils may include, for example, one or more of the following: almond oil, avocado oil, blackcurrant seed oil, brazil nut oil, canola oil, cashew oil, chia seed oil, coconut oil, corn oil, cottonseed oil, grapeseed oil, hazelnut oil, hemp seed oil, olive oil, palm oil, peanut oil, pecan oil, macadamia nut oil, perilla oil, pumpkin seed oil, rapeseed oil, safflower oil, seagrass oil, sea fennel oil, sesame oil, soybean oil, sunflower oil, walnut oil, and mixtures thereof. In some embodiments, the plant-derived oil is selected from the group consisting of coconut oil, avocado oil, olive oil, and mixtures thereof. In other embodiments, a plant-derived oil is coconut oil. In other embodiments, the plant-derived oil is avocado oil. In other embodiments, the plant-derived oil is olive oil. In other embodiments, a plant-derived oil is a mixture of olive oil and avocado oil. In other embodiments, a plant-derived oil is a mixture of olive oil and coconut oil. In other embodiments, a plant-derived oil is a mixture of avocado oil and coconut oil. In other embodiments, a plant-derived oil is a mixture of olive oil, avocado oil and coconut oil.

For example, a contemplated fatty acid composition that forms part of disclosed liquid compositions may have a weight ratio of omega-6 fatty acids:omega 9-fatty acids:omega-3 fatty acids of about 1:about 1.7 to about 2.3:about 5.7 to about 6.3. In other embodiments, the fatty acid composition has a weight ratio of omega-6 fatty acids:omega 9-fatty acids:omega-3 fatty acids of about 1:about 1.6 to about 2.4:about 5.6 to about 6.3. In other embodiments, the fatty acid composition has a weight ratio of omega-6 fatty acids:omega 9-fatty acids:omega-3 fatty acids of about 1:about 1.8 to about 2.2:about 5.8 to about 6.2. In other embodiments, the fatty acid composition has a weight ratio of omega-6 fatty acids:omega 9-fatty acids:omega-3 fatty acids of about 1:about 1.9 to about 2.1:about 5.9 to about 6.2. In other embodiments, the fatty acid composition has a weight ratio of omega-6 fatty acids:omega 9-fatty acids:omega-3 fatty acids of about 1:about 2.0 to about 2.2:about 6.0 to about 6.2. In other embodiments, the fatty acid composition has a weight ratio of omega-6 fatty acids:omega 9-fatty acids:omega-3 fatty acids of about 1:about 2.1 to about 2.2:about 6.1 to about 6.2. In other embodiments, the fatty acid composition has a weight ratio of omega-6 fatty acids:omega 9-fatty acids:omega-3 fatty acids of about 1:about 2.2:about 6.2. In other embodiments, the fatty acid composition has a weight ratio of omega-6 fatty acids:omega 9-fatty acids:omega-3 fatty acids of about 1:about 2.0:about 6.0. In other embodiments, the fatty acid composition has a weight ratio of omega-6 fatty acids:omega 9-fatty acids:omega-3 fatty acids of about 1:about 1.7 to about 2.3:about 3.0 to about 9.0, e.g., about 3.0, about 3.5, about 4.0, about 4.5, about 5.0, about 5.5, about 6.0, about 7.0, about 7.5, about 8.0, about 8.5, about 9.0.

A contemplated fatty acid composition that forms part of disclosed liquid compositions may include the omega-3 fatty acids eicosapentaenoic acid, docosahexaenoic acid, and α-linoleic acid. For example, in some embodiments the weight ratio of eicosapentaenoic acid:docosahexaenoic acid:α-linoleic acid is about 1:about 1.8 to about 2.2:about 3.5 to about 4.1. In other embodiments, the weight ratio of eicosapentaenoic acid:docosahexaenoic acid:α-linoleic acid is about 1:about 1.8 to about 2.2:about 3.6 to about 4.0. In other embodiments, the weight ratio of eicosapentaenoic acid:docosahexaenoic acid: α-linoleic acid is about 1:about 1.9 to about 2.1:about 3.7 to about 3.9. In other embodiments, the weight ratio of eicosapentaenoic acid:docosahexaenoic acid:α-linoleic acid is about 1:about 2.0:about 3.8. In other embodiments, the fatty acid composition component may include, for example, about 200 to about 1500 mg eicosapentaenoic acid, e.g., about 200 to about 1000 mg eicosapentaenoic acid, e.g., about 200 to about 500 mg eicosapentaenoic acid, e.g., about 200 to about 300 mg eicosapentaenoic acid, e.g., about 250 to about 30) mg eicosapentaenoic acid, e.g., about 282 mg eicosapentaenoic acid; about 450 to about 3000 mg docosahexaenoic acid; e.g., about 450 to about 2000 mg docosahexaenoic acid; e.g., about 450 to about 1000 mg docosahexaenoic acid: e.g., about 450 to about 750 mg docosahexaenoic acid; e.g., about 500 to about 600 mg docosahexaenoic acid; e.g., about 550 to about 600 mg docosahexaenoic acid; e.g., about 582 mg docosahexaenoic acid; and about 1000 to about 3000 mg α-linoleic acid, e.g., about 1000 to about 2000 mg α-linoleic acid, e.g., about 1000 to about 1500 mg α-linoleic acid, e.g., about 1000 to about 1200 mg α-linoleic acid, e.g., about 1067 mg α-linoleic acid.

A contemplated fatty acid composition that forms part of disclosed liquid compositions may further comprises vitamin D3 and/or vitamin E. For example, in other embodiments, the fatty acid composition further comprises vitamin D3. In other embodiments, the fatty acid composition further comprises vitamin E. In other embodiments, the fatty acid composition further comprises vitamin D3 and vitamin E. In other embodiments, the fatty acid composition comprises about 37.5 to about 125 mcg vitamin D3, e.g., about 40 to about 100 mcg vitamin D3, e.g., about 42.5 to about 75 mcg vitamin D3, e.g., about 45 to about 55 mcg vitamin D3, e.g., about 50 mcg vitamin D3; and vitamin E (e.g. about 8 mg vitamin E).

In some embodiments, a disclosed liquid composition has a shelf life of 3 months or more when stored at 4° C. In other embodiments, the liquid composition has a shelf life of 4 weeks or more when stored at 20° C. In other embodiments, the shelf life of the emulsion forming liquid composition is determined by metagenomic analysis. In other embodiments, the shelf life of the emulsion forming liquid composition is determined by culture analysis. In other embodiments, the shelf life of the emulsion forming liquid composition is determined by metagenomic analysis and culture analysis.

A contemplated emulsion-forming liquid composition may have a pH of about 3.2 to about 3.6 at 20° C. In other embodiments, the shelf-stable, emulsion-forming liquid composition has a pH of about 3.3 to about 3.5, e.g., 3.3, 3.4, 3.5, at 20° C. In other embodiments, the shelf-stable, emulsion-forming liquid composition has a pH of about 3.3 to about 3.8, e.g., 3.3, 3.4, 3.5, 3.6, 3.7, 3.8, at 20° C.

For example, disclosed herein is an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, phenylalanine, and tyrosine; and b) a fatty acid composition comprising at least three omega-3 fatty acids:wherein the aqueous liquid composition and the fatty acid composition form an amino acid/fatty acid buffer system allowing the creation of a fatty acid vesicle-based emulsion when the aqueous liquid composition and the fatty acid composition are shaken together.

In some embodiments, contemplated aqueous liquid composition portions of a disclosed liquid composition may include, for example, L-tryptophan and L-phenylalanine. In some embodiments, the L-tryptophan and L-phenylalanine are present in a weight ratio of about 1:about 1.7 to about 2.3. In other embodiments, the L-tryptophan and L-phenylalanine are present in a weight ratio of about 1:about 1.8 to about 2.2. In other embodiments, the L-tryptophan and L-phenylalanine are present in a weight ratio of about 1:about 1.9 to about 2.1. In other embodiments, the L-tryptophan and L-phenylalanine are present in a weight ratio of about 1:about 2.0. In some embodiments, a contemplated liquid beverage composition comprises, for example, about 150 to about 350 mg L-tryptophan, e.g., about 150 to about 300 mg L-tryptophan. e.g., about 150 to about 250 mg L-tryptophan. e.g., about 175 to about 225 mg L-tryptophan, e.g., about 200 mg L-tryptophan; and about 150 to about 450 mg L-phenylalanine, e.g., about 200 to about 450 mg L-phenylalanine, e.g., about 250 to about 450 mg L-phenylalanine, e.g., about 300 to about 450 mg L-phenylalanine, e.g., about 350 to about 450 mg L-phenylalanine, e.g., about 375 to about 425 mg L-phenylalanine, e.g., about 400 mg L-phenylalanine. In some embodiments a contemplated liquid composition comprising about 150 to 300 mg (e.g., about 200 mg) L-tyrosine also comprises about 37.5 to about 125 mcg (e.g., about 50 mcg) vitamin D3.

In other embodiments, contemplated fatty acid composition portions of a disclosed liquid composition may include, for example, eicosapentaenoic acid, docosahexaenoic acid, and α-linoleic acid. In some embodiments, the weight ratio of eicosapentaenoic acid:docosahexaenoic acid:α-linoleic acid is about 1:about 1.8 to about 2.2:about 3.5 to about 4.1. In other embodiments, the weight ratio of eicosapentaenoic acid:docosahexaenoic acid:α-linoleic acid is about 1:about 1.8 to about 2.2:about 3.6 to about 4.0. In other embodiments, the weight ratio of eicosapentaenoic acid:docosahexaenoic acid: α-linoleic acid is about 1:about 1.9 to about 2.1:about 3.7 to about 3.9. In other embodiments, the weight ratio of eicosapentaenoic acid:docosahexaenoic acid:α-linoleic acid is about 1:about 2.0:about 3.8. In other embodiments, the fatty acid composition component may include, for example, about 200 to about 1500 mg eicosapentaenoic acid, e.g., about 200 to about 1000 mg eicosapentaenoic acid, e.g., about 200 to about 500 mg eicosapentaenoic acid, e.g., about 200 to about 300 mg eicosapentaenoic acid, e.g., about 250 to about 300 mg eicosapentaenoic acid, e.g., about 282 mg eicosapentaenoic acid; about 450 to about 3000 mg docosahexaenoic acid; e.g., about 450 to about 2000 mg docosahexaenoic acid; e.g., about 450 to about 1000 mg docosahexaenoic acid; e.g., about 450 to about 750 mg docosahexaenoic acid; e.g., about 500 to about 600 mg docosahexaenoic acid; e.g., about 550 to about 600 mg docosahexaenoic acid; e.g., about 582 mg docosahexaenoic acid; and about 1000 to about 3000 mg α-linoleic acid, e.g., about 1000 to about 2000 mg α-linoleic acid, e.g., about 1000 to about 1500 mg α-linoleic acid, e.g., about 1000 to about 1200 mg α-linoleic acid, e.g., about 1067 mg α-linoleic acid.

In some embodiments, the fatty acid composition further comprises vitamin D3 and/or vitamin E. For example, in other embodiments, the fatty acid composition further comprises vitamin D3. In other embodiments, the fatty acid composition further comprises vitamin E. In other embodiments, the fatty acid composition further comprises vitamin D3 and vitamin E. In other embodiments, the fatty acid composition comprises about 37.5 to about 125 mcg vitamin D3, e.g., about 40 to about 100 mcg vitamin D3, e.g., about 42.5 to about 75 mcg vitamin D3, e.g., about 45 to about 55 mcg vitamin D3, e.g., about 50 mcg vitamin D3: and vitamin E (e.g. about 8 mg vitamin E).

A contemplated emulsion-forming liquid composition may further include magnesium and/or a salt or hydrate thereof. For example, in some embodiments the emulsion-forming liquid composition further comprises magnesium L-ascorbate dihydrate.

Also provided herein is a liquid beverage composition comprising L-tryptophan; L-phenylalanine; one or more omega-6 fatty acids; one or more omega-9 fatty acids; and three or more omega-3 fatty acids; wherein the weight ratio of omega-6 fatty acids:omega-9 fatty acids:omega-3 fatty acids is about 1:about 1.7 to about 2.3:about 5.7 to about 6.3; and an effective amount of a food-compatible acidic agent; wherein the liquid beverage composition has a pH of about 3.2 to about 3.6 as measured at 20° C.

Exemplary omega-3 fatty acids may include, for example, docosahexaenoic acid, eicosapentaenoic acid and α-linoleic acid, where for example the weight ratio of eicosapentaenoic acid:docosahexaenoic acid:α-linoleic acid is about 1:about 1.8 to about 2.2:about 3.5 to about 4.1, about 1:about 1.8 to about 2.2:about 3.6 to about 4.0, about 1.9 to about 2.1:about 3.7 to about 3.9, or about 1:about 2.0:about 3.8. The liquid beverage composition may further include, for example, magnesium L-ascorbate dihydrate. The liquid beverage composition may also include, for example, vitamin D3 and vitamin E.

In some embodiments the food-compatible acidic agent is citric acid. Such a food-compatible acidic agent may be in amount such that the liquid or beverage has pH of about 3.2 to about 3.6, e.g., 3.2, 3.3, 3.5, 3.6, at 20° C.; or about 3.3 to about 3.5, e.g., 3.3, 3.4, 3.5, at 20° C.; or about 3.2 to about 3.8, e.g., 3.2, 3.3, 3.4, 3.5, 3.6, 3.7, 3.8, at 20° C. The citric acid may be present in the form of a fruit juice, as described above. For example, the citric acid is present in the form of orange juice and/or lemon juice. For example, the citric acid is present in the form of orange juice and lemon juice.

Also provided herein is liquid beverage composition comprising flaxseed oil, algae oil, a plant-derived oil, L-tryptophan, and L-phenylalanine wherein for example, the omega-6 fatty acids, omega-9 fatty acids and omega-3 fatty acids present in the flaxseed oil, algae oil, and coconut oil are in a weight ratio of omega 6 fatty acids:omega 9 fatty acids:omega 3 fatty acids of about 1:about 1.7 to about 2.3:about 5.7 to about 6.3. For example, a liquid beverage composition is provided comprising: flaxseed oil, algae oil, a plant-derived oil, L-tryptophan, and L-phenylalanine:wherein the eicosapentaenoic acid, docosahexaenoic acid, and α-linoleic acid present in the flaxseed oil and algae oil combined are present in a weight ratio of eicosapentaenoic acid:docosahexaenoic acid:α-linoleic acid of about 1:about 1.8 to about 2.2:about 3.5 to about 4. Such a composition may further comprise citric acid in an amount whereby the liquid beverage composition has a pH of about 3.2 to about 3.6 at 20° C., e.g., 3.2, 3.3, 3.4, 3.5, 3.6, at 20° C. In other embodiments, the liquid beverage composition further comprises citric acid in an amount whereby the liquid beverage composition has a pH of about 3.3 to about 3.5 at 20° C. e.g., 3.3, 3.4, 3.5, at 20° C. In other embodiments, the liquid beverage composition further comprises citric acid in an amount whereby the liquid beverage composition has a pH of about 3.3 to about 3.8, e.g., 3.3, 3.4, 3.5, 3.6, 3.7, 3.8, at 20° C.

In some embodiments, the L-tryptophan and L-phenylalanine are present in a weight ratio of about 1:about 1.7 to about 2.3. In other embodiments, the L-tryptophan and L-phenylalanine are present in a weight ratio of about 1:about 1.8 to about 2.2. In other embodiments, the L-tryptophan and L-phenylalanine are present in a weight ratio of about 1:about 1.9 to about 2.1. In other embodiments, the L-tryptophan and L-phenylalanine are present in a weight ratio of about 1:about 2.0.

A fatty acid vesicle-forming liquid composition that may, for example, increase neurosystem uptake of amino acids present in the fatty acid vesicle-forming liquid composition when consumed by a subject, is also provided that comprises per 60 ml of the liquid composition: 1-5 ml flaxseed oil; about 1-5 ml algae oil; about 1-5 ml of a plant-derived oil, wherein the plant-derived oil is selected from the group consisting of olive oil, avocado oil, coconut oil, and mixtures thereof:about 200 to about 800 mg phenylalanine, e.g., about 150 to about 650 mg phenylalanine, e.g., about 150 to about 550 mg phenylalanine, e.g., 200 to about 600 mg phenylalanine, e.g., about 350 to about 450 mg phenylalanine, e.g., 400 mg L-phenylalanine; about 150 to about 450 mg tryptophan, e.g., about 150 to about 350 mg tryptophan, e.g., about 150 to about 250 mg tryptophan, about 175 mg to about 225 mg tryptophan, e.g., 200 mg L-tryptophan; and about 47 to about 59 ml, e.g., about 54 ml of a food-compatible aqueous solution with a pH of about 3.0 to about 3.9 at 20° C.; wherein when the liquid composition is shaken for e.g., 10 seconds or longer, (e.g., about 10, 20, 30, 40 seconds or more) the creation of fatty acid vesicles occurs, thereby effecting increased neurosystem uptake of the amino acids present in the liquid composition when consumed by the subject.

The fatty-acid vesicle-forming liquid composition may further comprise, per 60 ml of the liquid composition about 50 (or, e.g., 40-60) mcg vitamin D3 (e.g., 23 mcg) and/or about 8 mg vitamin E; about 200 (or, e.g., 150-250) mg α-lipoic acid and/or about 200 (or, e.g., 150-250) mg resveratrol and/or about 750 (or, e.g., 700-800) mg creatine; and optionally about 8 mg of zinc and/or about 326 (or e.g., 200-350) mg vitamin C and/or about 19 mg magnesium.

A fatty acid vesicle-forming liquid composition that may, for example, increase neurosystem uptake of amino acids present in the fatty acid vesicle-forming liquid composition when consumed by a subject, is also provided that comprises per 60 ml of the liquid composition:about 2 ml flaxseed oil; about 2 ml algae oil; about 2 ml of a plant-derived oil, wherein the plant-derived oil is selected from the group consisting of olive oil, avocado oil, coconut oil, and mixtures thereof; about 400 mg L-phenylalanine:about 200 mg L-tryptophan:and about 54 ml of a food-compatible aqueous solution with a pH of about 3.0 to about 3.9 at 20° C.; wherein when the liquid composition is shaken for 10 seconds or longer, the creation of fatty acid vesicles occurs, thereby effecting increased neurosystem uptake of the amino acids present in the liquid composition when consumed by the subject.

In some embodiments, the plant-derived oil is olive oil. In other embodiments, the plant-derived oil is avocado oil. In other embodiments, the plant-derived oil is coconut oil. In other embodiments, the plant-derived oil is a mixture of olive oil and avocado oil. In other embodiments, the plant-derived oil is a mixture of olive oil and coconut oil. In other embodiments, the plant-derived oil is a mixture of avocado oil and coconut oil. In other embodiments, the plant-derived oil is a mixture of olive oil, avocado oil and coconut oil.

In some embodiments, the food-compatible aqueous solution comprises, per 60 ml of the liquid composition:about 52 ml orange juice; and about 2 ml lemon juice.

The algae oil present in the fatty acid vesicle-forming liquid composition may be, for example, Schizochytrium sp, oil.

Also provided herein, for example, is method of enhancing brain performance in a human subject in need thereof, comprising administering to the human subject an effective amount of a composition disclosed herein, for example, a disclosed emulsion-forming liquid composition, a disclosed liquid beverage composition, or a disclosed fatty acid vesicle-forming liquid composition.

Further disclosed herein, for example, is a method of increasing energy levels, productivity, and/or creativity in a human subject in need thereof, comprising administering to the human subject an effective amount of a composition disclosed herein, for example, a disclosed, emulsion-forming liquid composition, a disclosed liquid beverage composition, or a disclosed fatty acid vesicle-forming liquid composition.

### EXAMPLES

The features and other details of the disclosure will be more readily understood by reference to the following examples, which are included merely for purposes of illustration of certain aspects and embodiments of the present disclosure, and are not intended to limit the invention in any way. No animals were used in the following examples.

### Example 1: Composition

A liquid composition of the present disclosure was prepared according to Table 1.

TABLE 1

| | Type | Amount | Unit | Active ingredient | Amount | Unit |
|---|---|---|---|---|---|---|
| Freshly pressed Orange | Liquid | 52 | ml | | | |
| juice | | | | | | |
| Lemon juice | Liquid | 2 | ml | Citric acid | | |
| Flaxseed oil | Oil | 2 | ml | Alpha-Linoleic acid | 1067 | mg |
| | | | | Omega-6 | 260 | mg |
| | | | | Omega-9 | 360 | mg |
| | | | | Vitamin E | 1 | mg |
| Algae oil | Oil | 2 | ml | Eicosapentaenoic acid | 282 | mg |
| | | | | Docosahexaenoic acid | 564 | mg |
| | | | | Other Omega-3 fatty | 188 | mg |
| | | | | acids | | |
| | | | | Omega-6 | 20 | mg |
| | | | | Omega-9 | 220 | mg |
| | | | | Vitamin E | 4 | mg |
| Coconut oil | Oil | 2 | ml | Omega-6 | 60 | mg |
| | | | | Omega-9 | 150 | mg |
| | | | | Medium-chain | 1800 | mg |
| | | | | triglycerides | | |
| Vitamin D3 oil | Oil | 0.1 | ml | Vitamin D3 | 50 | mcg |
| Vitamin E oil | Oil | 0.05 | ml | Vitamin E | 8 | mg |
| Magnesium L- | Solid | 345 | mg | Vitamin C | 326 | mg |
| ascorbate dihydrate | | | | | | |
| | | | | Magnesium | 19 | mg |
| L-Phenylalanine | Solid | 400 | mg | L-Phenylalanine | 400 | mg |
| L-Tryptophane | Solid | 200 | mg | L-Tryptophane | 200 | mg |
| Alpha-Lipoic acid | Solid | 200 | mg | Alpha-Lipoic acid | 200 | mg |
| Resveratrol | Solid | 200 | mg | Resveratrol | 200 | mg |
| Creatine | Solid | 750 | mg | Creatine | 750 | mg |
| pH Total | 3,2-3,6 | | | | | |
| High pressure | 2 cycles time 5 min | | | | | |
| processing | | | | | | |
| Shelf-life | >3 months | | 4° C. | | | |
| Shelf-life | >2 month | | 20° C. | | | |
| Omega-3:-6:-9 ratio | | | | | | Ratio |
| | | | | ALA | 1067 | |
| | | | | DHA | 564 | |
| | | | | EPA | 282 | |
| | | | | Other Omega-3 | 188 | |
| | | | | Total Omega-3 | 2101 | 6 |
| | | | | Total Omega-6 | 340 | 1 |
| | | | | Total Omega-9 | 730 | 2 |

### Example 2: Microbial Stability and Shelf Life

The chemical stability of a disclosed liquid composition stored at 22° C. was evaluated over five months. The results are shown in Table 2. The microbial stability and shelf life was also evaluated. A liquid composition was subjected to high pressure processing at 6000 bar for two cycles of 5 minutes. The treatment provided a shelf-life of at least 3 months at 4° C. or 22° C. as confirmed by metagenome analysis, culture analysis and colony count, with no significant changes in levels of e.g., lactic acid bacteria. Enterobacteriaceae, E. coli, yeast, mold, listeria and salmonella. The pH increased from 3.87 at day 1 to 4.47 at 3 months.

TABLE 2

| | | Day 1 | 5 Months |
|---|---|---|---|
| C 8:0 (MCT) | g/100 g | 22.01 | 22.48 |
| C 10:0 (MCT) | g/100 g | 15.49 | 15.93 |
| C 16:0 | g/100 g | 8.78 | 8.58 |
| C 18:0 | g/100 g | 1.87 | 1.74 |
| C 18:1 cis (omega 9) | g/100 g | 9.56 | 9.39 |
| C 18:2 cis | g/100 g | 5.52 | 5.39 |
| C 18:3 cis (alpha linoleic acid) | g/100 g | 17.87 | 17.74 |
| C 20:5 cis (eicosapentaenoic acid) | g/100 g | 5.66 | 5.56 |
| C 22:5 | g/100 g | 1.21 | 1.21 |
| C 22:6 (docosapentaenoic acid) | g/100 g | 10.36 | 10.21 |
| Sum saturated fatty | g/100 g | 3.50 | 4.10 |
| acids (in oil phase) | | | |
| Sum single-unsaturated | g/100 g | 0.69 | 0.79 |
| fatty acids (in oil phase) | | | |
| Sum poly-unsaturated | g/100 g | 2.90 | 3.40 |
| fatty acids (in oil phase) | | | |
| Tryptophan | % | 0.37 | 0.34 |
| Phenylalanine | % | 0.59 | 0.72 |
| Vitamin C | mg/kg | 5,934.00 | 4,967.00 |
| Vitamin D3 | μg/100g | 40.00 | 66.30 |
| alpha-Tocopherol | mg/100 g | 101.00 | 93.00 |
| delta-Tocopherol | mg/100 g | 9.15 | 9.01 |
| gamma-Tocopherol | mg/100 g | 43.22 | 40.68 |
| Vitamin E | mg/100 g | 112.70 | 104.00 |
| Zinc | mg/kg | 2.27 | 0.88 |
| Magnesium | mg/100 g | 431.50 | 458.20 |

### Example 3: Pharmacological Studies

Using the liquid composition according to Table 1, changes in energy level, productivity, and creativity, as well as changes in sleep quality, mood and well-being in a subject population were investigated as follows.

### Study Design

The subject population consisted of fifteen males ranging from 20 to 35 years old who were not using SSRI's, not using blood pressure lowering medications, and had no history of high blood pressure. Before commencing the 14-day study period, subjects were requested to disclose their use, amount, and type of any stimulating beverages and/or nutritional supplements; their use, amount and type of any stimulating medical and/or recreational drugs; and their use, amount and type of any sleep medication. The nutritional/dietary preference of each subject was also noted (e.g., no limitations, vegan, vegetarian, lactose-free or gluten-free diet). Subjects were requested to maintain their normal nutrition/dietary preferences for the duration of the study period.

Subjects were allocated to three tests groups. Subjects who used high levels of caffeine as a stimulating agent on a regular basis (>15 cups of coffee, espresso, green tea per day) were allocated to Group A (7 subjects). Subjects who used medical amphetamine medication (Adderall) as a stimulating agent on a regular basis were assigned to Group B (3 subjects). Subjects who did not use any stimulating agent on a regular basis were assigned to Group C (Control Group, 5 subjects). All participants worked in a job as a web or app designer or developer/coder.

For the first seven days of the study period (week 1), subjects in all study groups received 60 ml of a placebo beverage (orange juice with 6 ml wheat germ oil) daily. Subjects in Group A maintained their regular use of caffeine. Subjects in Group B maintained their regular use of Adderall. Subjects in Group C used no stimulating agent (control group).

For the next seven days of the study period (week 2), subjects in all study groups received 60 ml of the liquid composition according to Table 1 daily instead of the placebo beverage. Subjects in Groups A and B discontinued using their respective stimulating agent.

Throughout the course of the 14-day study period, each subject was challenged with a high-intensity, high-pressure, and potentially stressful work project. The work project was the same for each subject. At the end of each day, each subject completed a self-assessment questionnaire related to brain performance. Performance metrics included in the questionnaire were, for example, (i) number of nights with sleep without using sleeping pills; (ii) energy level at work during the working day: (iii) productivity at work or outside work; and (iv) creativity at work or outside work.

### Results

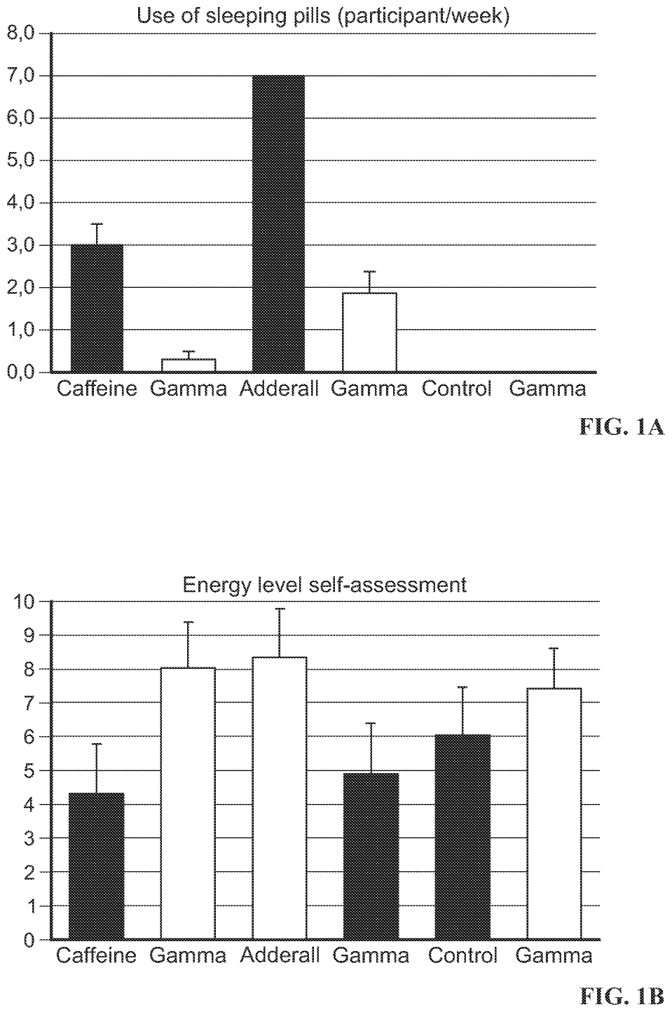

As shown in FIG. 1A, a significant reduction in the use of sleeping pills in the group using caffeine or Adderall (week 1) compared to replacement with the drink formula (week 2) caffeine or Adderall. Data shown are number of sleeping pills per participants per week.

As shown in FIG. 1B, a strong trend for higher energy level was observed in subjects consuming the drink formula (week 2) as a replacement for caffeine (week 1). Data shown are average self-assessment ratings in a range from 10 (highest energy level)-1 (lowest energy level). A trend for lower energy level was observed in subjects consuming the drink formula (week 2) as a replacement for Adderall (week 1). No apparent changes were observed in the control group.

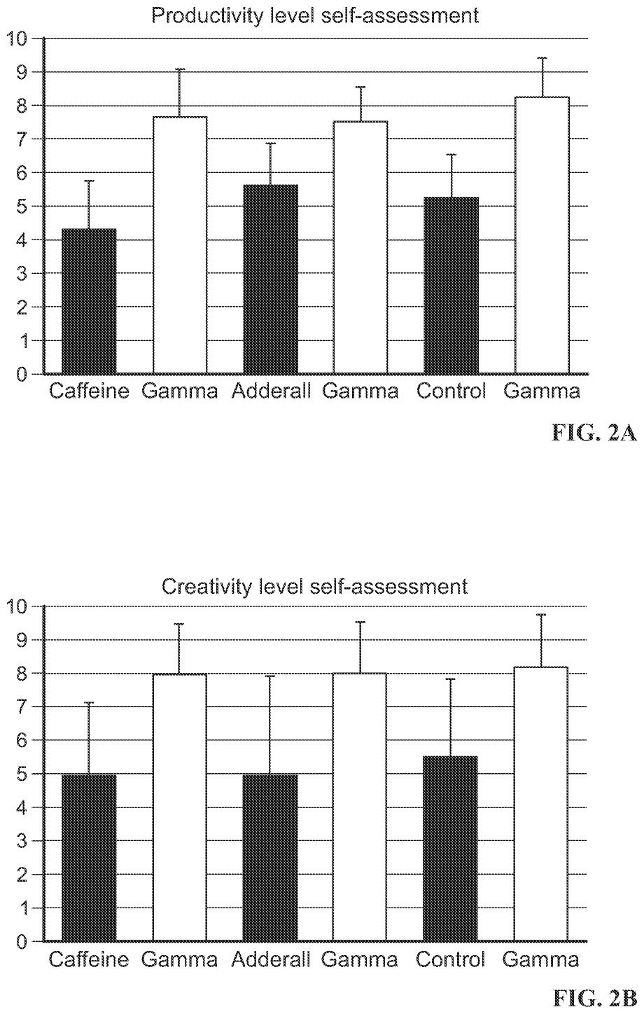

As shown in FIG. 2A, a positive trend for productivity level was observed in all groups, with the highest productivity level observed in subjects consuming the drink formula (week 2) as a replacement for caffeine (week 1). Data shown are average self-assessment ratings in a range from 10 (highest productivity level)-1 (lowest productivity level).

FIG. 2B shows the results for self-assessment of creativity level. On a group level no statistically significant effect of the drink formula on creativity was observed. A high error bar was observed, likely driven by strong positive impact of the drink formula on subjects with a generally lower creativity level in week 1. In contrast, subjects with a higher creativity level in week 1 observe lower positive impact by the drink formula. Data shown are average self-assessment ratings in a range from 10 (highest)-1 (lowest).

### Example 3: Pharmacological Studies

The effects of a disclosed composition in drink form on energy level, productivity and creativity in a subject population is investigated as follows.

For the first seven days of the study period (week 1), subjects in all study groups consume a placebo drink daily. Subjects were then randomized into four groups. For the next seven days of the study (week 2), Group D consume a drink formula containing all ingredients, Group NO consume a drink formula with no oil ingredients, Group NM consume a drink formula with no micronutrient ingredients, and Group P consume a placebo drink. The drink formulae per treatment group are shown in Table 3.

TABLE 3
Drink formulae per treatment group

| | Group D | Group NO | Group NM | Group P |
|---|---|---|---|---|
| flaxseed oil | + | − | + | − |
| algae oil | + | − | + | − |
| coconut oil | + | − | + | − |
| MCT oil | + | − | − | − |
| wheat germ oil | − | − | − | + |
| sunflower oil | − | − | − | + |
| phenylalanine | + | + | − | − |
| tryptophan | + | + | − | − |
| vitamin C | + | + | − | − |
| vitamin E | + | + | − | − |
| vitamine D3 | + | + | − | − |
| magnesium | + | + | − | − |
| orange juice | + | + | + | + |
| lemon juice | + | + | + | + |

Throughout the course of the 14-day study period, each subject is challenged with a high-intensity, high-pressure, and potentially stressful work project. The work project is the same for each subject. At the end of each day, each subject completes a self-assessment questionnaire related to brain performance. Performance metrics included in the questionnaire are, for example, (i) energy level at work during the working day; (ii) productivity at work or outside work; and (iii) creativity at work or outside work.

Group D reports significant improvement over baseline in energy level, productivity levels and creativity level (each self-reported scales from 0-5) compared to all other groups. No apparent effect is observed in groups drinking formula without omega-3 oils (NO), without micronutrients (NM) or placebo (P).

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. An emulsion-forming liquid composition, comprising:
an aqueous liquid composition having a pH of about 3.0 to about 3.9 as measured at 20° C., comprising at least two different amino acids each independently selected from the group consisting of tryptophan, 5-hydroxytryptophan, phenylalanine, and tyrosine; and
a fatty acid composition comprising at least one omega-9 fatty acid, at least one omega-6 fatty acid, and at least three omega-3 fatty acids;
wherein the aqueous liquid composition and the fatty acid composition form an amino acid/fatty acid buffer system allowing the creation of a fatty acid vesicle-based emulsion when the aqueous liquid composition and the fatty acid composition are shaken together.

2. The emulsion-forming liquid composition of claim 1, wherein the aqueous liquid composition and the fatty acid composition are contained within one container.

3. The emulsion-forming liquid composition of claim 1, wherein the aqueous liquid composition and the fatty acid composition are contained within separate containers before the emulsion-forming liquid composition is shaken.

4. The emulsion-forming liquid composition of any one of claims 1-3, wherein when consumed in emulsion form by a subject, provides increased neurosystem uptake of amino acids in the subject.

5. The emulsion-forming liquid composition of any one of claims 1-4, wherein the aqueous liquid composition further comprises a fruit juice.

6. The emulsion-forming liquid composition of claim 5, wherein the fruit juice comprises orange and/or lemon juice.

7. The emulsion-forming liquid composition of any one of claims 1-6, wherein the amino acids present in the aqueous liquid composition may each independently be an L-isomer or a mixture of D- and L-isomers.

8. The emulsion-forming liquid composition of any one of claims 1-7, wherein the aqueous liquid composition comprises L-tryptophan and L-phenylalanine.

9. The emulsion-forming liquid composition of any one of claims 1-8, further comprising magnesium and/or a salt or hydrate thereof.

10. The emulsion-forming liquid composition of any one of claims 1-9, further comprising magnesium L-ascorbate dihydrate.

11. The emulsion-forming liquid composition of any one of claims 1-10, further comprising one or more compounds each independently selected from the group consisting of α-lipoic acid, resveratrol, and/or creatine.

12. The emulsion-forming liquid composition of any one of claims 1-11, wherein the fatty acid composition comprises eicosapentaenoic acid, docosahexaenoic acid, and α-linoleic acid.

13. The emulsion-forming liquid composition of claim 12, wherein the weight ratio of eicosapentaenoic acid:docosahexaenoic acid:α-linoleic acid is about 1:about 1.8 to about 2.2:about 3.5 to about 4.1.

14. The emulsion-forming liquid composition of any one of claims 1-13, wherein the fatty acid composition has a weight ratio of omega-6 fatty acids:omega 9-fatty acids:omega-3 fatty acids of about 1:about 1.7 to about 2.3:about 5.7 to about 6.3.

15. The emulsion-forming liquid composition of any one of claims 1-14, wherein the fatty acid composition comprises flaxseed oil, algae oil, and a plant-derived oil selected from the group consisting of coconut oil, avocado oil, olive oil, and mixtures thereof.

16. The emulsion-forming liquid composition of any one of claims 1-15, wherein the fatty acid composition further comprises vitamin D3 and/or vitamin E.

17. The emulsion-forming liquid composition of any one of claims 1-16, wherein the liquid composition has a shelf life of 3 months or more when stored at 4° C. as determined by metagenomic analysis and/or culture analysis.

18. The emulsion-forming liquid composition of any one of claims 1-17, wherein the liquid composition has a shelf life of 8 weeks or more when stored at 20° C. as determined by metagenomic analysis and/or culture analysis.

19. The emulsion-forming liquid composition of any one of claims 1-18, wherein the shelf-stable, emulsion-forming liquid composition has a pH of about 3.2 to about 3.6 at 20° C.

20. An emulsion-forming liquid composition, comprising:
an aqueous liquid composition having a pH of about 3.0 to about 3.9 as measured at 20° C., comprising at least two different amino acids each independently selected from the group consisting of tryptophan, 5-hydroxytryptophan, phenylalanine, and tyrosine; and
a fatty acid composition comprising at least three omega-3 fatty acids;
wherein the aqueous liquid composition and the fatty acid composition form an amino acid/fatty acid buffer system allowing the creation of a fatty acid vesicle-based emulsion when the aqueous liquid composition and the fatty acid composition are shaken together.

21. The emulsion-forming liquid composition of claim 20, wherein the aqueous liquid composition comprises L-tryptophan and L-phenylalanine.

22. The liquid beverage composition of claim 21, wherein the L-tryptophan and L-phenylalanine are present in a weight ratio of about 1:about 1.8 to about 2.2.

23. The emulsion-forming liquid composition of any one of claims 20-23, wherein the fatty acid composition comprises eicosapentaenoic acid, docosahexaenoic acid, and α-linoleic acid.

24. The emulsion-forming liquid composition of claim 23, wherein the weight ratio of eicosapentaenoic acid:docosahexaenoic acid:α-linoleic acid is about 1:about 1.8 to about 2.2:about 3.5 to about 4.1.

25. A liquid beverage composition comprising:
one or more omega-6 fatty acids;
one or more omega-9 fatty acids;
three or more omega-3 fatty acids;
wherein the weight ratio of omega-6 fatty acids:omega-9 fatty acids:omega-3 fatty acids is about 1:about 1.7 to about 2.3:about 5.7 to about 6.3;
L-tryptophan;
L-phenylalanine; and
an effective amount of a food-compatible acidic agent; wherein the liquid beverage composition has a pH of about 3.2 to about 3.6 as measured at 20° C.

26. The liquid beverage composition of claim 25, wherein the food-compatible acidic agent is citric acid.

27. The liquid beverage composition of claim 26, wherein the citric acid is present in the form of a combination of orange juice and lemon juice.

28. The liquid beverage composition of any one of claims 25-27, further comprising vitamin D and/or vitamin E.

29. The liquid beverage composition of any one of claims 25-28, wherein the three or more omega-3 fatty acids comprises docosahexaenoic acid, eicosapentaenoic acid and α-linoleic acid.

30. The liquid beverage composition of any one of claims 25-29, further comprising magnesium L-ascorbate.

31. The emulsion-forming liquid composition of any one of claims 25-30, wherein the fatty acid composition further comprises α-lipoic acid, resveratrol and/or creatine.

32. A liquid beverage composition comprising:
flaxseed oil, algae oil, a plant-derived oil, L-tryptophan, and L-phenylalanine;
wherein the omega-6 fatty acids, omega-9 fatty acids and omega-3 fatty acids present in the flaxseed oil, algae oil, and plant derived oil are in a weight ratio of omega 6 fatty acids:omega 9 fatty acids:omega 3 fatty acids of about 1:about 1.7 to about 2.3:
about 5.7 to about 6.3.

33. A liquid beverage composition comprising:
flaxseed oil, algae oil, a plant-derived oil, L-tryptophan, and L-phenylalanine;
wherein the eicosapentaenoic acid, docosahexaenoic acid, and α-linoleic acid present in the flaxseed oil, algae oil, and plant-derived oil combined are present in a weight ratio of eicosapentaenoic acid:docosahexaenoic acid:α-linoleic acid of about 1:about 1.8 to about 2.2:about 3.5 to about 4.1.

34. The liquid beverage composition of claim 33, wherein the L-tryptophan and L-phenylalanine are present in a weight ratio of about 1:about 1.8 to about 2.2.

35. The liquid beverage composition of claim 33 or 34, further comprising citric acid in an amount whereby the liquid beverage composition has a pH of about 3.2 to about 3.6 at 20° C.

36. The liquid beverage composition of any one of claims 33-35, wherein the plant-derived oil is selected from the group consisting of coconut oil, avocado oil, olive oil, and mixtures thereof.

37. The liquid beverage composition of any one of claims 33-36, wherein the plant-derived oil is coconut oil.

38. A fatty acid vesicle-forming liquid composition comprising, per 60 ml of the liquid composition:
about 1-5 ml flaxseed oil;
about 1-5 ml algae oil;
about 1-5 ml of a plant-derived oil, wherein the plant-derived oil is selected from the group consisting of olive oil, avocado oil, coconut oil, and mixtures thereof;
about 150 to about 650 mg L-phenylalanine;
about 150 to about 450 mg L-tryptophan; and
about 47-59 ml of a food-compatible aqueous solution with a pH of about 3.0 to about 3.9 at 20° C.;
wherein when the liquid composition is shaken for 10 seconds or longer, the creation of fatty acid vesicles occurs.

39. A fatty acid vesicle-forming liquid composition comprising, per 60 ml of the liquid composition:
about 2 ml flaxseed oil;
about 2 ml algae oil;
about 2 ml of a plant-derived oil, wherein the plant-derived oil is selected from the group consisting of olive oil, avocado oil, coconut oil, and mixtures thereof;
about 400 mg L-phenylalanine;
about 200 mg L-tryptophan; and
about 54 ml of a food-compatible aqueous solution with a pH of about 3.0 to about 3.9 at 20° C.;
wherein when the liquid composition is shaken for 10 seconds or longer, the creation of fatty acid vesicles occurs.

40. The fatty acid vesicle-forming liquid composition of claim 39, further comprising, per 60 ml of the liquid composition:
about 50 mcg vitamin D3 and/or about 8 mg vitamin E;
about 200 mg α-lipoic acid and/or about 200 mg resveratrol and/or about 750 mg creatine; and
optionally about 19 mg magnesium and/or about 326 mg vitamin C.

41. The fatty acid vesicle-forming liquid composition of claim 39 or 40, wherein the food-compatible aqueous solution comprises, per 60 ml of the liquid composition:
about 52 ml orange juice; and
about 2 ml lemon juice.

42. The fatty acid vesicle-forming liquid composition of any one of claims 39-41, wherein the algae oil is Schizochytrium sp, oil.

43. A method of enhancing brain performance in a human subject in need thereof, comprising administering to the human subject an effective amount of a composition of any one of claims 1-42.

44. A method of increasing energy levels, productivity, and/or creativity in a human subject in need thereof, comprising administering to the human subject an effective amount of a composition of any one of claims 1-42.
